(19) United States
(12) Patent Application Publication
(10) Pub. No.: US 2019/0135903 A1
(43) Pub. Date: May 9, 2019

(54) IDENTIFYING AND TREATING SUBPOPULATIONS OF PAROXYSMAL NOCTURNAL HEMOGLOBINURIA (PNH) PATIENTS

(71) Applicant: Alexion Pharmaceuticals, Inc., New Haven, CT (US)

(72) Inventor: Camille Bedrosian, Woodbridge, CT (US)

(21) Appl. No.: 15/560,606

(22) PCT Filed: Mar. 29, 2016
(86) PCT No.: PCT/US2016/024624

§ 371 Date: Sep. 22, 2017

(51) Int. Cl. C07K 16/18; C07K 16/40; A61P 7/00; A61K 9/00

(52) U.S. Cl. CPC C07K 16/18; C07K 16/40; A61K 38/00; A61K 9/0019; A61P 7/00

(57) ABSTRACT

Provided herein are materials and methods that identify a population of treatment-responsive PNH patients, particularly those who can be effectively treated anti-C5 antibody, or antigen binding fragment thereof, such as eculizumab.

### BACKGROUND

Paroxysmal Nocturnal Hemoglobinuria (PNH) is a chronic and debilitating disease caused by an acquired genetic mutation in hematopoietic stem cells in the bone marrow that results in the loss of complement regulatory proteins (CD55 and CD59) on the surface of red blood cells (RBCs), platelets and white blood cells (WBCs). The central mechanism of PNH is uncontrolled complement activity, which leads to chronic intravascular hemolysis and platelet activation. One outcome of hemolysis is a reduction in the total number of RBCs, and anemia results when the rate of hemolysis outpaces RBC production. The release of cell-free hemoglobin from hemolyzed RBCs directly leads to consumption of nitric oxide (NO), which causes vascular and other smooth muscle constriction and platelet activation, resulting in thrombosis, chronic kidney disease (CKD), pulmonary hypertension and end-organ damage in patients with PNH. While patients with hemolysis may not exhibit overtly severe clinical symptoms at presentation or between paroxysms, their PNH-related complications continue to worsen in the presence of chronic hemolysis as evidenced by increased morbidities and mortality over time.

Effective treatment of PNH patients is based on inhibition of the complement component C5. A commercially available antibody therapy, eculizumab, which targets C5, is a life-changing therapeutic treatment for patients with PNH. As there is a benefit from diagnosing and treating PNH patients early, and there is a need to identify patients who would benefit from treatment even in the absence of severe symptoms, materials and methods useful for identifying one or more subpopulations of treatment-responsive patients is needed.

### SUMMARY

The present invention is based, at least in part, on the discovery that PNH patients can be identified as candidates to be treated with, for example, an anti-C5 antibody, or antigen binding fragment thereof, such as eculizumab, irrespective of a lack of traditional PNH symptoms (e.g., aplastic anemia, hemolytic anemia, iron-deficiency anemia), hemorrhagic symptoms, jaundice, thrombosis or embolism, infection, neurologic symptoms, hemoglobinuria, hemolytic anemia, marrow failure, thrombophilia, vasomotor tone, dyspnea, nitric oxide depletion, gastrointestinal symptoms (e.g., abdominal pain), backache, chronic kidney disease, dyspnea, dysphagia, chest pain, erectile dysfunction and fatigue) and/or the patient's transfusion history. The data described herein show that patients with lactate dehydrogenase (LDH) concentration of LDH≥1.5×ULN (upper limit of normal), and without a history of PNH symptoms and/or transfusion have a substantial burden of disease and are at increased risk of morbidities and mortality. Among the patients treated with eculizumab, a clinically and statistically significant reduction in hemolysis, as indicated by normalization of LDH levels, and improvement in clinical symptoms are demonstrated. These data support a favorable benefit/risk profile for administering an anti-C5 antibody, or antigen binding fragment thereof, to patients who have an lactate dehydrogenase (LDH) concentration of LDH≥1.5×ULN, without a history of PNH symptoms.

An exemplary anti-C5 antibody is eculizumab (Souris®) comprising the heavy and light chains having the sequences shown in SEQ ID NOs:10 and 11, respectively, or antigen binding fragments and variants thereof. In other embodiments, the antibody comprises the heavy and light chain complementarity determining regions (CDRs) or variable regions (VRs) of antibody BNJ441. Accordingly, in one embodiment, the antibody comprises the CDR1, CDR2, and CDR3 domains of the heavy chain variable (VH) region of antibody BNJ441 having the sequence shown in SEQ ID NO:7, and the CDR1, CDR2 and CDR3 domains of the light chain variable (VL) region of antibody BNJ441 having the sequence shown in SEQ ID NO:8. In another embodiment, the antibody comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5, and 6, respectively. In another embodiment, the antibody comprises VH and VL regions having the amino acid sequences set forth in SEQ ID NO:7 and SEQ ID NO:8, respectively.

Another exemplary anti-C5 antibody is antibody BNJ441 (also known as ALXN1210) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of antibody BNJ441. Accordingly, in one embodiment, the antibody comprises the CDR1, CDR2, and CDR3 domains of the heavy chain variable (VH) region of antibody BNJ441 having the sequence shown in SEQ ID NO:12, and the CDR1, CDR2 and CDR3 domains of the light chain variable (VL) region of antibody BNJ441 having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively. In another embodiment, the antibody comprises VH and VL regions having the amino acid sequences set forth in SEQ ID NO:12 and SEQ ID NO:8, respectively.

In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In one embodiment, the disclosure is directed to a method of reducing intravascular hemolysis, comprising administering an effective amount of an anti-C5 antibody, or antigen binding fragment thereof, to a patient who has paroxysmal nocturnal hemoglobinuria, wherein prior to treatment the patient is determined to have high disease activity as determined by a lactate dehydrogenase concentration of about ≥1.5×ULN, and wherein the patient does not exhibit traditional PNH symptoms (e.g., aplastic anemia, hemolytic anemia, iron-deficiency anemia), hemorrhagic symptoms, jaundice, thrombosis or embolism, infection, neurologic symptoms, hemoglobinuria, hemolytic anemia, marrow failure, thrombophilia, vasomotor tone, dyspnea, nitric oxide depletion, gastrointestinal symptoms (e.g., abdominal pain), backache, chronic kidney disease, dyspnea, dysphagia, chest pain, erectile dysfunction and fatigue)). In a particular embodiment, the patient is determined to have a lactate dehydrogenase concentration of about 1.5× to about 3.5×ULN. In a particular embodiment, the patient had never had a blood transfusion.

In another embodiment, the disclosure is directed to methods of reducing intravascular hemolysis in a human patient having PNH, the method comprising administering an effective amount an anti-C5 antibody, or antigen binding fragment thereof, comprising CDR1, CDR2, and CDR3 heavy chain sequences as set forth in SEQ ID NOs:1, 2, and 3, respectively, and CDR1, CDR2, and CDR3 light chain sequences as set forth in SEQ ID NOs:4, 5, and 6, respectively, to the patient patient, wherein prior to treatment the patient is determined to have high disease activity as determined by a lactate dehydrogenase concentration of about ≥1.5× upper limit of normal (ULN), and wherein the patient does not exhibit symptoms of PNH prior to treatment.

In another embodiment, the disclosure is directed to methods of reducing intravascular hemolysis in a human patient having PNH, the method comprising administering an effective amount an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, to the patient patient, wherein prior to treatment the patient is determined to have high disease activity as determined by a lactate dehydrogenase concentration of about ≥1.5× upper limit of normal (ULN), and wherein the patient does not exhibit symptoms of PNH prior to treatment.

In another embodiment, the disclosure is directed to methods of reducing intravascular hemolysis in a human patient having PNH, the method comprising administering an effective amount an anti-C5 antibody, or antigen binding fragment thereof, comprising CDR1, CDR2, and CDR3 heavy chain sequences as set forth in SEQ ID NOs:19, 18, and 3, respectively, CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, to the patient, wherein prior to treatment the patient is determined to have high disease activity as determined by a lactate dehydrogenase concentration of about ≥1.5× upper limit of normal (ULN), and wherein the patient does not exhibit symptoms of PNH prior to treatment.

In another embodiment, the disclosure is directed to methods of reducing intravascular hemolysis in a human patient having PNH, the method comprising:
(i) selecting a patient who has a lactate dehydrogenase concentration of about ≥1.5× upper limit of normal (ULN) and does not exhibit symptoms of PNH prior to treatment, from a subpopulation of PNH patients; and
(ii) administering an anti-C5 antibody, or antigen binding fragment thereof, comprising CDR1, CDR2, and CDR3 heavy chain sequences as set forth in SEQ ID NOs:1, 2, and 3, respectively, and CDR1, CDR2, and CDR3 light chain sequences as set forth in SEQ ID NOs:4, 5, and 6, respectively, to the patient.

In another embodiment, the disclosure is directed to methods of reducing intravascular hemolysis in a human patient having PNH, the method comprising:
(i) selecting a patient who has a lactate dehydrogenase concentration of about ≥1.5× upper limit of normal (ULN) and does not exhibit symptoms of PNH prior to treatment, from a subpopulation of PNH patients; and
(ii) administering an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, to the patient.

In another embodiment, the disclosure is directed to methods reducing intravascular hemolysis in a human patient having PNH, the method comprising:
(i) selecting a patient who has a lactate dehydrogenase concentration of about ≥1.5× upper limit of normal (ULN) and does not exhibit symptoms of PNH prior to treatment, from a subpopulation of PNH patients; and
(ii) administering an anti-C5 antibody, or antigen binding fragment thereof, comprising CDR1, CDR2, and CDR3 heavy chain sequences as set forth in SEQ ID NOs:19, 18, and 3, respectively, CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, to the patient.

In a particular embodiment, the patient does not have a history of thrombosis or fatigue.

### Patientpatientpatient.

In a particular embodiment, treatment begins with an initial phase comprising administering 600 mg of an anti-C5 antibody, or antigen binding fragment thereof, once a week for 4 weeks. In a particular embodiment, the initial phase of treatment is followed by a maintenance phase comprising administering 900 mg of an anti-C5 antibody, or antigen binding fragment thereof, during the fifth week. In a particular embodiment, the maintenance phase is followed by administration of 900 mg of an anti-C5 antibody, or antigen binding fragment thereof, every 14±2 days. In a particular embodiment, the patient is a pediatric patient having a body weight of between about 30 and about 40 kg, and treatment begins with an initial phase comprising administering 600 mg of an anti-C5 antibody, or antigen binding fragment thereof, once a week for 2 weeks. In a particular embodiment, the initial phase of treatment is followed by a maintenance phase comprising administering 900 mg of an anti-C5 antibody, or antigen binding fragment thereof, during the third week. In a particular embodiment, the maintenance phase is followed by administration of 900 mg of an anti-C5 antibody, or antigen binding fragment thereof, every 2 weeks.

In a particular embodiment, the patient is a pediatric patient having a body weight of between about 20 and about 30 kg, and treatment begins with an initial phase comprising administering 600 mg of an anti-C5 antibody, or antigen binding fragment thereof, once a week for 2 weeks. In a particular embodiment, the initial phase of treatment is followed by a maintenance phase comprising administering 600 mg of an anti-C5 antibody, or antigen binding fragment thereof, during the third week. In a particular embodiment, the maintenance phase is followed by administration of 600 mg of an anti-C5 antibody, or antigen binding fragment thereof, every 2 weeks. In a particular embodiment, the patient is a pediatric patient having a body weight of between about 10 and about 20 kg, and treatment begins with an initial phase comprising administering 600 mg of an anti-C5 antibody, or antigen binding fragment thereof, once a week for 1 week. In a particular embodiment, the initial phase of treatment is followed by a maintenance phase comprising administering 300 mg of an anti-C5 antibody, or antigen binding fragment thereof, during the second week. In a particular embodiment, the maintenance phase is followed by administration of 300 mg of an anti-C5 antibody, or antigen binding fragment thereof, every 2 weeks. In a particular embodiment, the patient is a pediatric patient having a body weight of between about 5 and about 10 kg, and treatment begins with an initial phase comprising administering 300 mg of an anti-C5 antibody, or antigen binding fragment thereof, once a week for 1 week. In a particular embodiment, the initial phase of treatment is followed by a maintenance phase comprising administering 300 mg of an anti-C5 antibody, or antigen binding fragment thereof, during the second week. In a particular embodiment, the maintenance phase is followed by administration of 300 mg of an anti-C5 antibody, or antigen binding fragment thereof, every 3 weeks.

The efficacy of the treatment methods provided herein can be assessed using any suitable means. In one embodiment, the treatment results in terminal complement inhibition. In another embodiment, the treatment results in a reduction of hemolysis as assessed by lactate dehydrogenase (LDH) levels. In one embodiment, the patient experiences a return to normal lactate dehydrogenase concentration within six months of treatment with the anti-C5 antibody, or antigen binding fragment thereof. In another embodiment, the treatment produces a shift toward normal levels of a hemolysis-related hematologic biomarker selected from the group consisting of free hemoglobin, haptoglobin, reticulocyte count, PNH red blood cell (RBC) clone and D-dimer. In another embodiment, the treatment produces a reduction in major adverse vascular events (MAVEs). In another embodiment, the treatment produces a shift toward normal levels of a chronic disease associated biomarker selected from the group consisting estimated glomerular filtration rate (eGFR) and spot urine:albumin:creatinine and plasma brain natriuretic peptide (BNP). In another embodiment, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale.

The anti-C5 antibodies, or antigen binding fragments thereof, can be administered to a patient by any suitable means. In one embodiment, the antibodies are formulated for intravenous administration.

### DETAILED DESCRIPTION

Various definitions are used throughout this document. Most words have the meaning that would be attributed to those words by one skilled in the art. Words specifically defined either below or elsewhere in this document have the meaning provided in the context of the present invention as a whole and as are typically understood by those skilled in the art. For example, as used herein, the singular forms “a,” “an” and “the” include plural references unless the content clearly dictates otherwise. Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. In case of conflict, the present specification, including definitions, will control.

Provided herein are materials and methods useful for identifying a population of patients who are responsive to treatment for paroxysmal nocturnal hemoglobinuria (PNH), e.g., via an anti-C5 antibody, or antigen binding fragment thereof, such as eculizumab. Described herein are methods for determining the concentration of particular analyte(s), e.g., lactate dehydrogenase (LDH), wherein a patient's LDH concentration is indicative of the patient's condition with respect to PNH and/or PNH-associated morphologies, and is also indicative of the patient's likely responsiveness to a PNH treatment regimen. Evidence of clinical benefit of the treatment was unexpectedly found to not be limited to patients with PNH with history of transfusion, but, as shown herein is also demonstrated in patients with no prior history of transfusion and/or a lack of traditional PNH symptoms (e.g., aplastic anemia, hemolytic anemia, iron-deficiency anemia), hemorrhagic symptoms, jaundice, thrombosis or embolism, infection, neurologic symptoms, hemoglobinuria, hemolytic anemia, marrow failure, thrombophilia, vasomotor tone, dyspnea, nitric oxide depletion, gastrointestinal symptoms (e.g., abdominal pain), backache, chronic kidney disease, dyspnea, dysphagia, chest pain, erectile dysfunction and fatigue). Any of the aspects described below can be used in any combination or with any other elements known in the art.

As used herein, the term “patient” or “patient” is a human patient (e.g., a patient having Paroxysmal Nocturnal Hemoglobinuria (PNH)).

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. An effective amount can be administered in one or more administrations.

As used herein, the terms “initial phase”, “induction” and “induction phase” are used interchangeably and refer to the first phase of treatment in the clinical trial.

As used herein, the terms “maintenance” and “maintenance phase” are used interchangeably and refer to the second phase of treatment in the clinical trial. In certain embodiments, treatment is continued as long as clinical benefit is observed or until unmanageable toxicity or disease progression occurs.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the anti-C5 antibody, or antigen binding fragment thereof,).

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered by a protein engineering technique (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial or engineered polypeptide constructs which comprise at least one antibody-derived antigen binding site.

The anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

Anti-C5 antibodies (or VH/VL domains derived therefrom) suitable for use in the invention can be generated using methods well known in the art. Alternatively, art recognized anti-C5 antibodies can be used. Antibodies that compete with any of these art-recognized antibodies for binding to C5 also can be used.

An exemplary anti-C5 antibody is eculizumab (Souris®; Alexion Pharmaceuticals, Inc., Cheshire, Conn.), or an antibody that binds to the same epitope on C5 as or competes for binding to C5 with eculizumab (See, e.g., Kaplan (2002) Curr Opin Investig Drugs 3(7):1017-23; Hill (2005) Clin Adv Hematol Oncol 3(11):849-50; and Rother et al. (2007) Nature Biotechnology 25(11):1256-1488). Soliris®, is a formulation of eculizumab which is a recombinant humanized monoclonal IgG2/4κ antibody produced by murine myeloma cell culture and purified by standard bioprocess technology. Eculizumab contains human constant regions from human IgG2 sequences and human IgG4 sequences and murine complementarity-determining regions grafted onto the human framework light- and heavy-chain variable regions. Eculizumab is composed of two 448 amino acid heavy chains and two 214 amino acid light chains and has a molecular weight of approximately 148 kDa. Eculizumab comprises the heavy and light chain amino acid sequences set forth in SEQ ID NOs: 10 and 11, respectively; heavy and light chain variable region amino acid sequences set forth in SEQ ID NOs: 7 and 8, respectively; and heavy chain variable region CDR1-3 and light chain variable region CDR1-3 sequences set forth in SEQ ID NOs: 1, 2, and 3 and 4, 5, and 6, respectively.

Another exemplary anti-C5 antibody is antibody BNJ441 comprising heavy and light chains having the sequences shown in SEQ ID NOs:14 and 11, respectively, or antigen binding fragments and variants thereof. BNJ441 (also known as ALXN1210) is described in PCT/US2015/019225 and U.S. Pat. No. 9,079,949, the teachings or which are hereby incorporated by reference. BNJ441 is a humanized monoclonal antibody that is structurally related to eculizumab (Soliris®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441. Accordingly, in one embodiment, the antibody comprises the CDR1, CDR2, and CDR3 domains of the VH region of BNJ441 having the sequence set forth in SEQ ID NO:12, and the CDR1, CDR2 and CDR3 domains of the VL region of BNJ441 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO: 8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO: 8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., PCT/US2015/019225 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q311I, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in BNJ441 and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable an anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

The anti-C5 antibodies, or antigen binding fragments thereof, can be administered to a patient by any suitable means. In one embodiment, the antibodies are formulated for intravenous administration.

LDH is a marker of intravascular hemolysis (Hill, A. et al., Br. J. Haematol., 149:414-25, 2010; Hillmen, P. et al., N. Engl. J. Med., 350:552-9, 2004; Parker, C. et al., Blood, 106:3699-709, 2005). RBCs contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):359 Abs.0903, 2008; Kanakura, Y. et al., Int. J. Hematol., 93:36-46, 2011).

As shown herein, the correlation between cell-free hemoglobin and LDH concentration is important for monitoring, diagnosing and treating PNH patients—in particular with regard to identifying PNH patients who will respond to treatment, e.g., eculizumab. Cell-free hemoglobin and LDH concentration are measured, for example, during subsequent monitoring of treated patients to determine the efficacy of treatment, e.g., in clinical trials comprising the clinical development program (TRIUMPH (Transfusion Reduction Efficacy and Safety Clinical Investigation Using Eculizumab in Paroxysmal Nocturnal Hemoglobinuria), and SHEPHERD (Safety in Hemolytic PNH Patients Treated with Eculizumab: A Multi-center Open-label Research Design Study), E05-001). As described herein, LDH concentration obtained at baseline and then serially throughout a treatment period, is an important measure of hemolysis. Baseline levels of cell-free plasma hemoglobin are highly elevated in patients with PNH with LDH≥1.5-fold above the upper limit of normal (LDH≥1.5×ULN), with a significant correlation between LDH and cell-free plasma hemoglobin (Hillmen, P. et al., N. Engl. J. Med., 355:1233-43, 2006).

The disclosure is directed to, in part, identifying a subpopulation of PNH patients who exhibit an LDH concentration above ULN and who are not being treated with an anti-C5 antibody, or antigen binding fragment thereof, such as eculizumab. The subpopulation can, for example, have an LDH concentration of about 1.5×ULN, at least about 1.5×ULN, greater than 1.5×ULN, about 1.5×ULN to about 3.5×ULN, about 1.5×ULN to about 5.0×ULN, about 1.5×ULN to about 7.5×ULN, about 1.5×ULN to about 10.0×ULN, about 1.5×ULN to about 15.0×ULN, about 2.5×ULN, at least about 2.5×ULN, greater than 2.5×ULN, about 2.5×ULN to about 5.0×ULN, about 2.5×ULN to about 7.5×ULN, about 2.5×ULN to about 10.0×ULN, about 2.5×ULN to about 15.0×ULN, about 3.5×ULN, at least about 3.5×ULN, greater than about 3.5×ULN, about 3.5×ULN to about 5.0×ULN, about 3.5×ULN to about 7.5×ULN, about 3.5×ULN to about 10.0×ULN or about 3.5×ULN to about 15.0×ULN. As used herein, the term “about” refers to ±20%, ±10% or ±5% of a value.

Described herein are data that show that particular subpopulations of PNH patients, even such patients who do not exhibit otherwise severe PNH symptoms, can be effectively treated. Such treatment, e.g., with eculizumab, a modified eculizumab, a variant eculizumab or a functional eculizumab analog, is effective in treating symptoms that are manifested and in prophylactically treating the patient against more severe PNH symptoms and manifestations. Early treatment prevents degradation of the patient's condition to the point of needing transfusions. Symptoms include, but are not limited to, for example, hemolysis, anemia (e.g., aplastic anemia, hemolytic anemia, iron-deficiency anemia), hemorrhagic symptoms, jaundice, thrombosis or embolism, infection, neurologic symptoms, hemoglobinuria, hemolytic anemia, marrow failure, thrombophilia, vasomotor tone, dyspnea, nitric oxide depletion, gastrointestinal symptoms (e.g., abdominal pain), backache, chronic kidney disease, dysphagia, erectile dysfunction and fatigue. PNH-related symptoms can be, for example, a result of PNH or a treatment for PNH. The symptoms associated with PNH can be manifested as severe, mild or not manifested. One of skill in the art would recognize that, provided with the data described herein, a subpopulation of PNH patients who are identified to have an LDH concentration higher than the ULN, exhibit PNH-associated morphologies or are at risk for exhibiting such morphologies. Although previous studies had indicated a need for, for example, eculizumab in patients with a history of having blood transfusions, described herein are data indicating that eculizumab treatment is effective in populations that have no history of transfusions and/or a lack of traditional PNH symptoms, as long as they exhibit elevated levels of LDH.

As shown herein, for example, in patients with LDH≥1.5×ULN who have not received eculizumab, there is substantial risk of major adverse vascular event (MAVE) regardless of transfusion history, consistent with the fact that it is the underlying pathophysiology of the disease (i.e., complement-mediated intravascular hemolysis), which is the fundamental cause of the increased risk of thrombotic events (TE) associated with PNH. Treating the underlying cause of the disease, by blocking complement activation with an anti-C5 antibody, or antigen binding fragment thereof, such as eculizumab, directly reduces this risk as evident by the significantly reduced rate of MAVE.

LDH concentration can be measured, for example, in various samples obtained from a patient, in particular, serum samples. As used herein, the term “sample” refers to biological material from a patient. Although serum LDH concentration is of interest, samples can be derived from other sources, including, for example, single cells, multiple cells, tissues, tumors, biological fluids, biological molecules or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are known in the art and can be readily adapted to obtain a sample that is compatible with the method utilized.

Data described herein have indicated, contrary to previous interpretations and label usage for eculizumab, that PNH treatment is effective in PNH patients even in cases where the patient does not have a history of transfusions and/or does not exhibit traditional PNH symptoms. Using LDH concentration as an indicator of treatment responsiveness and efficacy, the Examples below show that in PNH patients who exhibit ≥1.5×ULN are responsive to, for example, eculizumab treatment, as demonstrated by improvements in, for example, LDH concentration, reduction of hemolysis, improved RBC count and improvement in fatigue and related PNH symptoms. Materials and methods described herein, therefore, are useful for identifying a treatment-responsive subpopulation of PNH patients, independent of previous transfusion history and/or independent of classic PNH symptoms.

Patientpatientpatient

A patient's (patient's) medical history can be informative as to treatment efficacy. A PNH patient, for example, can exhibit a variety of PNH-related effects. A patient, for example, can have a history of thrombosis, meaning the patient has had a thrombotic event at least once and likely more than once in the past. The patient's treatment history can also be informative, as, for example, a patient can have a history of treatment for PNH that included, for example, regular or single-event blood transfusions. A patient with a history of blood transfusions, therefore, is one who has had at least one and likely more than one or a regular schedule of blood transfusions. A history can be for the patient's lifetime, or for a defined period of time (e.g., within about 1 month, within about three months, within about six months, within about 9 months, within about 1 year, within about 2 years or within about 5 years of a specific time point, e.g., an expected treatment or diagnosis date).

The efficacy of the treatment methods provided herein can be assessed using any suitable means. In one embodiment, the treatment produces at least one therapeutic effect selected from the group consisting of a reduction or cessation in fatigue, abdominal pain, dyspnea, dysphagia, chest pain, and erectile dysfunction. In another embodiment, the treatment results in terminal complement inhibition. In another embodiment, the treatment results in a reduction of hemolysis as assessed by lactate dehydrogenase (LDH) levels. In another embodiment, the treatment produces a shift toward normal levels of a hemolysis-related hematologic biomarker selected from the group consisting of free hemoglobin, haptoglobin, reticulocyte count, PNH red blood cell (RBC) clone and D-dimer. In another embodiment, the treatment produces a reduction in major adverse vascular events (MAVEs). In another embodiment, the treatment produces a shift toward normal levels of a chronic disease associated biomarker selected from the group consisting estimated glomerular filtration rate (eGFR) and spot urine:albumin:creatinine and plasma brain natriuretic peptide (BNP). In another embodiment, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale.

### EXAMPLES

The invention is further described in the following examples, which do not limit the scope of the invention described in the claims.

### Example 1. PNH: Disease of Chronic Hemolysis with Severe Consequences

Data were evaluated from one large prospective observational study and several interventional clinical trials of eculizumab in patients with PNH elevated hemolysis, as measured by LDH concentration (LDH≥1.5×ULN) as an indicator of severity of disease.

Analysis of patients with PNH enrolled in the ongoing observational prospective study known as the International PNH Registry (M07-001; hereinafter “PNH Registry”), which specifically includes patients with LDH≥1.5×ULN at baseline, with and without a history of transfusion prior to baseline (see study criteria for NCT01374360 at the clinicaltrials.gov web site). The present analysis demonstrates that patients with PNH without a history of transfusion suffer from PNH-related morbidities to an extent similar to that observed in patients with transfusions. Patients with PNH who were treated with eculizumab had a statistically significant and clinically meaningful improvement in PNH-related symptoms compared with patients with PNH patients who did not receive eculizumab and had no history of transfusion. In patients treated with eculizumab, notably, LDH levels returned to normal or near normal levels at about six months, whereas LDH levels remained elevated in the “No Eculizumab” group.

The PNH Registry is an international non-interventional study conducted in over 20 countries including Europe, Japan, USA and Canada that continues to enroll patients and accrue longitudinal follow-up. It is the largest prospective data collection study in patients with PNH that also evaluates patient outcomes, including the long-term safety of eculizumab. It serves as an important source for documenting the burden of disease including morbidity and mortality, clinical outcomes and progression of PNH in patients treated with eculizumab and patients not treated with eculizumab—irrespective of transfusion history. Clinical and laboratory data also provide the basis for the assessment of key efficacy parameters such as degree of hemolysis and quality of life.

The PNH registry was used to evaluate the efficacy of eculizumab in PNH patients with no history of RBC transfusion. These patients had high disease activity as defined by elevated hemolysis (LDH≥1.5×ULN) and the presence of any one of the following related clinical symptom(s) like: fatigue, hemoglobinuria, abdominal pain, shortness of breath (dyspnea), anemia (hemoglobin <100 g/L), major adverse vascular event (including thrombosis), dysphagia or erectile dysfunction.

In the PNH Registry, patients treated with eculizumab were observed to have a reduction in hemolysis and associated symptoms. At 6 months, patients treated with eculizumab with no history of RBC transfusion had significantly (p<0.001) reduced LDH levels (median LDH of 305 U/L; Table 4). Furthermore, 74% of the patients treated with eculizumab experienced clinically meaningful improvements in FACIT-Fatigue score (i.e., increase by 4 points or more) and 84% in EORTC fatigue score (i.e., decrease by 10 points or more). No changes were observed in these symptoms in the cohort not treated with eculizumab.

An additional analysis was performed using the same population of patients. The inclusion of one additional year of data collection yielded results (e.g., changes in LDH, abdominal pain, backache, dysphagia, erectile dysfunction, hemoglobinuria, hemolysis, etc.).

To further support the clinically meaningful benefit and safety of eculizumab treatment in patients with PNH with hemolysis closer to the critical threshold of LDH≥1.5×ULN, a post-hoc analysis was completed for a subset of patients with LDH between 1.5×ULN to 3.5×ULN without a history of transfusion. The results of this subpopulation analysis are comparable to those of the full analysis population. Median LDH values in this subset of the eculizumab-treated group decreased from 740 U/L at baseline to 332.5 U/L at 6 months (n=14), compared to the “No Eculizumab” subset (758.5 U/L vs 719.5 U/L, respectively, n=58). A substantial improvement in fatigue was also observed in the eculizumab-treated subset (median change of +10.0 for FACIT-Fatigue (Functional Assessment of Chronic Illness Therapy) and −22.2 for EORTC fatigue (European Organisation for Research and Treatment of Cancer)), in contrast to the No Eculizumab subset, which demonstrated a median change of 0.0 in both scales. Consistent with the total population of patients analyzed in the PNH Registry analysis, patients with an LDH ratio between 1.5×ULN to 3.5×ULN are at considerable risk for thrombotic events (TE) (rate of 7.70 TE per 100-patient years from study enrollment to initiation of eculizumab). Following treatment with eculizumab, the rate of TE for this same subset of patients decreased to 0 per 100-patient years. The safety profile of this subset of patients is consistent with the profile of eculizumab in the entire eculizumab-treated group in the full analysis. These results support the relevance of the role of eculizumab treatment in providing a clinically meaningful benefit and a favorable benefit/risk profile for patients with PNH across the spectrum of hemolysis as measured by LDH concentration.

Overall, no new safety signals were observed in an updated safety analysis in patients in the eculizumab-treated group. The No Eculizumab group, in fact, had higher rates of infections, impaired renal function, pulmonary hypertension, hemolysis and death relative to the eculizumab-treated group.

The clinically meaningful and positive benefit/risk profile of eculizumab treatment in patients without a history of transfusion is comparable to that demonstrated in other clinical trials. TRIUMPH (C04-001), a randomized, double-blind registrational prospective placebo-controlled study for eculizumab in PNH (TRIUMPH (C04-001)), enrolled patients with LDH≥1.5×ULN. These patients also had to have had a history of ≥4 transfusions in the 12 months prior to enrollment. Eculizumab treatment resulted in a statistically significant and sustained reduction in hemolysis as demonstrated by reduction in LDH level and improvement in fatigue, global health, all five EORTC functioning scales, pain and dyspnea. Eculizumab also was shown to statistically significantly reduce free hemoglobin and the need for transfusions. These study results support a favorable benefit/risk profile in this heavily transfused population (Brodsky, R. et al., Blood, 111:1840-7, 2008).

SHEPHERD (C04-002), an open-label, non-comparative study, enrolled patients with LDH≥1.5×ULN. This study allowed patients with less intensive transfusion history, with an inclusion criterion of a history of ≥1 transfusions in the 24 months prior to enrollment. The study included patients with 0-1 transfusions in the 12 months prior to enrollment. Similar to the results observed in TRIUMPH, eculizumab treatment resulted in statistically significant reduction in hemolysis as demonstrated by LDH level. Statistically significant improvement in fatigue, global health, all five EORTC functioning scales, pain, dyspnea, appetite loss, insomnia, nausea, vomiting and diarrhea were also observed. Eculizumab also significantly reduced free hemoglobin. Consistent with the results from TRIUMPH, these study results support a favorable benefit/risk profile in a broader patient population, including the 22 patients with 0-1 transfusions within the 12 months prior to enrollment.

The Extension Trial (E05-001) was an extension study that enrolled patients from TRIUMPH and SHEPHERD and allowed for longitudinal analysis of the benefits of eculizumab treatment in these patients with PNH. Patients from the placebo arm of the TRIUMPH study initiated eculizumab treatment upon enrollment in the trial. Eculizumab treatment resulted in a statistically significant reduction in TE rate from 7.37 events per 100 patient years (124 total events) prior to receiving eculizumab treatment, compared to 1.07 events per 100 patient years in the same patients (3 total events) during eculizumab treatment (p<0.001) (Hillmen, P. et al., Blood, 110:4123-8, 2007). Patients in the placebo group had an event rate of 4.38 events per 100 patient years during the 26-week placebo treatment period. The importance of these findings reinforce that TE are unpredictable and are the leading cause of death in patients with PNH. Improvement in renal function was also observed during eculizumab therapy. These study results support a favorable long term treatment benefit/risk profile among a population of patients with LDH≥1.5×ULN at baseline and a mixed history of transfusions.

### LDH≥1.5×ULN is a Clinically Meaningful Threshold for Intravascular Hemolysis in Patients with PNH and an Independent Risk Factor for the Morbidities and Mortality Associated with PNH

The clinical trials comprising the clinical development program (TRIUMPH, SHEPHERD, the Extension Trial) all enrolled patients with PNH with LDH≥1.5×ULN. These studies all demonstrate a profound benefit of eculizumab treatment and a positive benefit/risk profile. The results from the prospectively defined analysis of data from the PNH Registry also included only patients with LDH≥1.5×ULN, and demonstrate a positive benefit/risk profile with eculizumab treatment. Additional data published in the medical literature identifies LDH≥1.5×ULN as a significant risk factor for the morbidities and mortality associated with PNH. These data include an independent analysis of patients, with and without a history of transfusions, in the M07-001 PNH Registry (Schrezenmeier, H. et al., Haematologica, 99:922-9, 2014) as well as analyses based on a large retrospective study.

Historically, and prior to the availability of eculizumab therapy, patients with PNH suffered from premature mortality. The relevance of this LDH threshold in terms of mortality can be summarized as follows:
A significantly higher prevalence of mortality (14% vs. 4%; p=0.048) in patients with LDH≥1.5×ULN compared to patients with LDH<1.5×ULN (Kim, J. et al., Korean J. Hematol., 45:269-74, 2010).
Patients with LDH≥1.5×ULN at diagnosis had a 4.8-fold greater mortality rate compared to the age- and gender-matched general population (p<0.001). Patients with LDH<1.5×ULN had a similar mortality rate as the age- and gender-matched general population (p=0.824).
LDH≥1.5×ULN was an independent risk factor of mortality (multivariate odds ratio=10.57, 95% CI:(1.36, 81.93), p=0.024), and LDH≥1.5×ULN was significantly associated with premature mortality compared to LDH<1.5×ULN (univariate odds ratio 5.0; 95% CI (1.15, 21.70); p=0.009).
In contrast, a sensitivity analysis identified that higher LDH thresholds at diagnosis of ≥3.0×ULN (odds Ratio 1.8; 95% CI (0.78, 4.09); p=0.162) and ≥5.0×ULN (odds ratio 2.0; 95% CI (0.91, 4.32); p=0.082) compared to <3.0×ULN or <5.0×ULN, respectively, were not significant predictors of premature mortality.
Coexistence of aplastic anemia or other bone marrow disorders did not differ significantly between patients with LDH≥1.5×ULN and LDH<1.5×ULN.

Patients with PNH are at permanent thrombotic risk despite the absence of clinical evidence of TE. Approximately 60% of patients with PNH without clinically diagnosed TE have evidence of TE as measured by high-sensitivity MRI (Hill, A. et al., Blood, 107:2131-7, 2006). TE increases the relative risk of death in PNH by 5- to 10-fold (Nishimura J. et al., Medicine (Baltimore), 83:193-207, 2004; Socie, G. et al., Lancet, 348:573-7, 1996) and thrombosis is the leading cause of death in patients with PNH. Data that establish the importance of an LDH≥1.5×ULN as a risk factor for TE can be summarized as follows:
The risk of TE in patients with LDH≥1.5×ULN is 7× greater than for patients with LDH<1.5×ULN (Hillmen, P. et al., Br. J. Haematol., 162:62-73, 2013; Lee, J. et al., Int. J. Hematol., 97:749-57, 2013).
A significantly higher prevalence of thrombosis in patients with LDH≥1.5×ULN compared to patients with LDH<1.5×ULN (22% vs. 4%; p=0.003; 15.6% vs 8.4%; p<0.001).
Using a threshold of LDH≥1.5×ULN at diagnosis, 96% of patients with TE were detected. Using LDH≥3.0× or 5.0×ULN detected only 67% and 47% of patients with TE, respectively.
LDH≥1.5×ULN is associated with a higher prevalence of symptoms such as, for example, abdominal pain, chest pain and hemoglobinuria, which are all significant risk factors for TE.
Parameters such as age, bone marrow failure history, clone size, WBC count, platelet count and hemoglobin level were not significant risk factors for TE (P-value range: 0.111-0.981).

The prevalence of dyspnea, dysphagia and scleral icterus was significantly greater in patients with LDH≥1.5×ULN than in patients with LDH<1.5×ULN, and overall, 96% of patients with LDH≥1.5×ULN reported at least one symptom other than fatigue, whereas only 0.6% of patients with elevated LDH levels reported fatigue and no other symptoms. These data, collected from a population of patients that included patients with no reported history of transfusion, further support a threshold LDH level of ≥1.5×ULN as an important parameter for characterizing the burden of the disease.

These effects of hemolysis are independent of the presence or severity of anemia. While anemia may be a manifestation of hemolysis, the morbidities associated with the complement-mediated intravascular hemolysis of PNH have been shown in large part to be independent of the degree of anemia.

Clinical studies have demonstrated that fatigue in patients with PNH, which can be disabling, is due to intravascular hemolysis and is independent of changes in the severity of anemia. In an independent analysis of patients from the SHEPHERD and TRIUMPH studies, multivariate analysis indicates that reduction of hemolysis is predictive of improvement in fatigue independent of an improvement in anemia (1.07, p=0.028; (Hill, A., Clin. Adv. Hematol. Oncol., 6:499-500, 2008)). Similar results were found in a separate clinical study (Kanakura, Y. et al., Int. J. Hematol., 93:36-46, 2011). It is understood that complement-mediated NO depletion and ischemia contribute to severe fatigue in PNH through hemostatic activation, inflammation, renal insufficiency and pulmonary hypertension (Hill, A. et al., Br. J. Haematol., 149:414-25, 2010).

Dyspnea, a common symptom of pulmonary hypertension, affects 66% of patients with PNH. Improvement in dyspnea in patients with PNH is significantly and positively correlated with changes in measures of pulmonary hypertension due to intravascular hemolysis, independent of anemia. Dyspnea as assessed by EORTC QLQ-C30 was also found to be independent of anemia in an unrelated study, with significant (p=0.006) improvement occurring with eculizumab treatment in the absence of observable changes in hemoglobin levels. It is worth noting that patients studied all had LDH≥1.5×ULN.

Data from the PNH Registry analysis also demonstrate that among the 189 patients who had no history of transfusion (all had LDH≥1.5×ULN per the inclusion criteria), 15.3% had a history of major adverse vascular event (MAVE) with more than half of the events (16/29) occurring in the “No Eculizumab” group. These data indicate that the risk of TE is independent of the need for transfusions.

Together these data highlight that patients with LDH≥1.5×ULN are at risk for the morbidities and mortality associated with PNH, and this risk is independent of the need for transfusions.

Treatment with eculizumab in patients with PNH leads to rapid and sustained control of hemolysis as measured by LDH and a reduction in the morbidities associated with the disease. Eculizumab effectively prevents the intravascular hemolysis that results from complement activation by inhibiting terminal complement, thus it provides clinical benefit by directly addressing the root cause of symptoms (e.g., hemolysis) associated with PNH. This benefit is demonstrated in patients with PNH with elevated LDH (≥1.5×ULN) and independent of the need for transfusions and/or the presence or severity of anemia. While some patients with PNH with LDH<1.5×ULN may also experience a broad range of morbidities associated with PNH, it is an LDH≥1.5×ULN that is the threshold and independent risk factor for many of the serious and disabling morbidities associated with the disease, including TE, which is the leading cause of death in patients with PNH. Collectively, the data from the clinical trial program and the PNH Registry consistently demonstrate that treatment with eculizumab results in important clinical benefit for patients with PNH with LDH≥1.5×ULN.

### Example 2. Defining the Target Patient Population to Patients with PNH with LDH≥1.5×ULN

Given that the central mechanism of PNH is uncontrolled complement activation, which leads to chronic intravascular hemolysis and platelet activation (Hillmen, P. N. Engl. J. Med., 333:1253-8, 1995), the level of intravascular hemolysis provides a direct measure of disease burden in patients with PNH.

Clinical trials comprising the clinical development program (TRIUMPH, SHEPHERD, the Extension Trial) enrolled patients with PNH with an LDH≥1.5×ULN. Those studies consistently demonstrate a profound benefit of eculizumab treatment and a positive benefit/risk profile. Corresponding with the reduction in hemolysis to normal or near normal levels, benefits of eculizumab treatment included significant reduction in disabling fatigue, abdominal pain, dyspnea, dysphagia, erectile dysfunction and improved quality of life and measures of functioning. Longitudinal results from the Extension Trial importantly demonstrate significantly (p<0.0005) reduced risk in TE/MAVE in a time-matched analysis pre- and post-eculizumab, as well as time-dependent improvement in kidney function (Hillmen, P. et al., Br. J. Haematol., 162:62-73, 2013).

The presence of chronic intravascular hemolysis, as measured by LDH≥1.5×ULN, has also been demonstrated to be an independent risk factor for morbidities, including the risk of thrombosis, and mortality in patients with PNH. Higher LDH ratios have been found not to be independent risk factors. TE is the leading cause of death in patients with PNH, and it is known that TE increases the relative risk of death in PNH by 5- to 10-fold (Nishimura, J. et al., Medicine (Baltimore), 83:193-207, 2004). Multiple analyses have demonstrated that LDH≥1.5×ULN is an independent risk factor for TE.

LDH≥1.5×ULN is also an independent risk factor for mortality. While patients with PNH can exhibit anemia, anemia is not a demonstrated risk factor for the morbidities or mortality in patients with PNH. The etiology of anemia in PNH is multifactorial. It can arise from chronic intravascular hemolysis, and it arises from the underlying bone marrow disorders known to be associated with PNH (Kawaguchi, T. & Nakakuma, H., Int. J. Hematol., 86:27-32, 2007) that in turn lead to deficient RBC production. Chronic intravascular hemolysis, and not anemia, is the key risk factor for the morbidities of PNH. The coexistence of aplastic anemia or other bone marrow disorders has not been shown to differ significantly between patients with LDH≥1.5×ULN and LDH<1.5×ULN. LDH≥1.5×ULN is also associated with higher prevalence of symptoms such as abdominal pain, chest pain, hemoglobinuria, dyspnea, dysphagia, pulmonary hypertension and scleral icterus (Hill, A. et al., Br. J. Haematol., 158:409-14, 2012).

### Improved Survival in Eculizumab Treated Patients

A median survival for patients with PNH not treated with eculizumab is 10 to 20 years from the time of diagnosis, including 35% mortality at 5 years (de Latour, R. et al., Blood, 112:3099-106, 2008; Socie, G. et al., Lancet, 348:573-7, 1996). In contrast, survival of patients with PNH treated with eculizumab is comparable to age- and sex-matched normal controls (p=0.46; Kelly, R. et al., Blood, 117:6789-92, 2011). Given the natural history of the disease, and the unpredictability of TE (Hill, A. et al., Blood, 121:4985-96, 2013), which is the leading cause of death in patients with PNH, it is important to identify patients who will most benefit from eculizumab treatment. The data from clinical trials comprising the clinical development program, as well as the PNH Registry analysis and data in the literature, together demonstrate the burden of disease in patients with PNH with clinically meaningful intravascular hemolysis as measured by LDH≥1.5×ULN.

### No Specific Differences in Patient Characteristics Identified in the Eculizumab-Treated Group at Enrolment and at Baseline in the PNH Registry Analysis

To better understand and qualify the burden of disease in patients with PNH prior to eculizumab treatment, an analysis of patient characteristics in the eculizumab-treated (“Eculizumab”) group from the time of Registry enrollment to the start of eculizumab was performed. The analysis was limited to patients with a minimum of six months between enrollment and initiation of eculizumab. Table 1 summarizes the results.

TABLE 1
Characteristics of Patient with Minimum of Six Months Between
Registry Enrollments to Start of Eculizumab

| | Eculizumab | |
|---|---|---|
| | No Transfusions | |
| | N = 12 | |
| Any MAVE between enrollment and eculizumab | 2/12 | (16.7) |
| start, n/N | | |
| Any TE MAVE between enrolment and | 2/12 | (16.7) |
| eculizumab start, n/N | | |
| LDH, n | 10 | |
| LDH (U/L) at enrollment, median (min, max) | 858.0 | (207.0, 1679.0) |
| LDH (U/L) at eculizumab start, median (min, | 856.0 | (489.0, 2588.0) |
| max) | | |
| % change in LDH levels, median (min, max) | 31.7 | (−51.4, 136.2) |
| LDH Ratio (×ULN), n | 10 | |
| LDH ratio (×ULN) at enrollment, median (min, | 2.4 | (0.8, 6.7) |
| max) | | |
| LDH ratio (×ULN) at eculizumab start, median | 3.0 | (1.9,7.7) |
| (min, max) | | |
| % change in LDH ratio (×ULN), median (min, | 32.1 | (−12.5, 136.2) |
| max) | | |
| Clone size, n | 6 | |
| % GPI-deficient granulocytes at enrollment, | 56.1 | (12.9, 90.0) |
| median (min, max) | | |
| % GPI-deficient granulocytes at eculizumab | 64.4 | (12.8, 89.0) |
| start, median (min, max) | | |
| Percent change in clone size | 2.0 | (−68.1, 319.6) |
| Hemoglobin, n | 11 | |
| Hemoglobin (g/L) at enrollment, median (min, | 106.0 | (86.0, 127.0) |
| max) | | |
| Hemoglobin (g/L) at eculizumab start, median | 108.0 | (77.0, 138.0) |
| (min, max) | | |
| % change in hemoglobin levels, median (min, | 0.0 | (−17.2, 54.7) |
| max) | | |
| RBC, n | 10 | |
| RBC (×10¹²/L) at enrollment, median (min, max) | 3.2 | (2.4, 4.0) |
| RBC (×10¹²/L) at eculizumab start, median (min, | 3.3 | (2.8, 4.2) |
| max) | | |
| % change in RBC | −2.7 | (−15.8, 63.1) |

For the main analysis provided herein, patients in the Eculizumab group must have initiated eculizumab at the time of enrollment or after enrolling in the Registry. Start of eculizumab treatment was set as the baseline. As defined in the statistical analysis plan (ESAP), these patients did not have a transfusion for a minimum of six months prior to baseline. There are a limited number of patients (n=12) with data available due to the fact that the majority of patients in the Eculizumab group initiated treatment at or soon after enrolment. Given the small number of patients meeting these requirements, the data are limited. Nevertheless, an evaluation of the data that are available identifies no specific parameter or event and no trend in a parameter or event for initiating eculizumab treatment.

Table 2 summarizes the changes in LDH levels over time. There was one patient in the Eculizumab group and three patients in the No Eculizumab group added to the primary outcome analysis of LDH at six months. The Eculizumab group shows a profound and highly significant (p<0.001) reduction in LDH at six months, which was sustained over time. In sharp contrast, the LDH value in the No Eculizumab group showed little change from baseline at the six month time point. Although some reduction in LDH values are observed in the No Eculizumab group at 18 and 24 months, as indicated in Table 2, these elevated levels still represent LDH≥1.5-fold above the upper limit of normal (Table 3). A decrease in patient number is observed at later time points because there were fewer patients enrolled in the PNH Registry.

TABLE 2
PNH Registry Analysis - Changes in LDH (U/L) during follow up

| | Eculizumab | | | No Eculizumab | | |
|---|---|---|---|---|---|---|
| | No Transfusion | | | No Transfusion | | |
| | (N = 45) | | | (N = 144) | | |
| | | | Change | | | Change |
| | | | in N | | | in N |
| | | | from | | | from |
| | Median (min, | | 1 Jul. | Median (min, | | 1 Jul. |
| | max) | N | 2013 | max) | N | 2013 |
| Base- | 1431.0 (301.0, | 45 | 0 | 1095.5 (360.0, | 144 | 0 |
| line | 4661.0) | | | 4893.0) | | |
| 6 | 293.5 (142.0, | 38 | +1 | 1033.0 (237.0, | 102 | +3 |
| month^{a)} | 1497.0) | | | 5212.0) | | |
| 12 | 342.0 (172.0, | 31 | +5 | 1014.5 (240.0, | 102 | +13 |
| month | 1279.0) | | | 4879.0) | | |
| 18 | 295.0 (163.0, | 27 | +11 | 864.0 (231.0, | 77 | +21 |
| month | 1266.0) | | | 5229.0) | | |
| 24 | 340.0 (174.0, | 21 | +9 | 615.0 (232.0, | 55 | +16 |
| month | 779.0) | | | 4451.0) | | |

^{a)}Primary outcome

Table 3 summarizes the changes in LDH ratios over time. Median LDH ratios returned to normal or near normal levels in the Eculizumab group within six months and remained normal at all follow-up time points. In contrast, the No Eculizumab group median LDH levels remained elevated above 1.5×ULN at all time points, indicating the continued presence of intravascular hemolysis.

TABLE 3
PNH Registry Analysis - Changes in LDH ratio (ULN) during follow up

| | Eculizumab | | | | | |
|---|---|---|---|---|---|---|
| | No Transfusion | | | | | |
| | (N = 45) | | | No Eculizumab | | |
| | | | Change | No Transfusion | | |
| | | | in N | (N = 144) | | |
| | Median | | from | | | Change in |
| | (min, | | 1 Jul. | Median | | N from |
| | max) | N | 2013 | (min, max) | N | 1 Jul. 2013 |
| Baseline | 4.3 (1.5, | 45 | 0 | 3.4 (1.5, | 144 | 0 |
| | 15.8) | | | 11.4) | | |
| 6 month^{a)} | 1.0 (0.7, | 36 | +1 | 3.2 (0.9, | 97 | +1 |
| | 3.4) | | | 9.5) | | |
| 12 month | 1.1 (0.7, | 30 | +5 | 3.3 (1.0, | 93 | +8 |
| | 2.7) | | | 11.3) | | |
| 18 month | 1.1 (0.7, | 26 | +11 | 2.8 (0.7, | 71 | +17 |
| | 2.6) | | | 12.2) | | |
| 24 month | 1.0 (0.7, | 20 | +9 | 2.4 (0.9, | 53 | +15 |
| | 1.7) | | | 10.4) | | |

^{a)}Primary outcome

### Major Adverse Vascular Event (MAVE) or Hemoglobin is not a Reliable Marker to Refine the Patient Population

As summarized in Table 1, no new MAVE occurred between enrollment and initiation of eculizumab treatment, and median hemoglobin levels and RBC counts were stable. Median LDH values were unchanged from enrollment to initiation of eculizumab treatment (858 vs. 856 U/L, respectively). Median LDH ratios were elevated at enrolment (2.4×ULN). Although there was an increase to 3.0×ULN at eculizumab initiation, an LDH threshold of ≥3.0×ULN is not a more sensitive indicator of mortality than LDH≥1.5×ULN. There was a modest increase in median clone size, with a median change of 2%. While median clone size was higher at the start of eculizumab treatment than at the time of enrollment, the range in clone size was the same. Therefore, MAVE, hemoglobin levels and/or clone size are not adequate measures of the burden of disease.

Collectively, these data identify no specific parameter, event or trend for eculizumab treatment initiation.

### Example 3

Published guidelines advise that flow cytometry measuring ≥1% of WBC reduced or lacking GPI protein (PNH cells or PNH clone size ≥1%) confirms the diagnosis of PNH. The sensitivity of 1% is sufficient for detecting patients with clones associated with hemolytic and/or thrombotic PNH (Borowitz et al. 2010). Guidelines also recommend that PNH patients with clones <1% be routinely monitored for change in clinical signs and symptoms or change in clone size.

The PNH Registry allows for inclusion of any patient with a diagnosis of PNH or a detected PNH clone (minimum level of 0.01% PNH cells detected) to be enrolled. The inclusion criteria of the analysis for patients having a granulocyte clone size ≥1%, was selected to be consistent with the above referenced guidelines.

Similar Burden of Disease Between Type II Variation Subgroups

Although the PNH Registry does allow for a heterogeneous patient population to be enrolled, results from the analysis of the population of patients included in the Type II variation indicate that at baseline, all patients, irrespective of clone size or transfusion history, had a high burden of disease, which includes 7/144 (4.9%) patients in the never-treated cohort with <10% GPI-deficient granulocytes.

The median granulocyte clone was 66% (1, 100) for never-treated patients without a history of transfusion and 71% (13, 99) for treated patients without a history of transfusion. All patients included in the analysis were required to have elevated hemolysis (LDH≥1.5×ULN) at baseline. The median LDH value of never-treated patients without a history of transfusion was 1431 U/L (range 301, 4661), while that of the treated patients without a history of transfusion was 1096 U/L (range 360, 4893). Additionally, patients without a history of transfusion presented with clinically meaningful signs and symptoms of PNH including severe fatigue, reduction in quality of life and history of major adverse vascular events (MAVE).

Based on the similarities in the baseline granulocyte clone size and other clinical characteristics, there is no evidence that there is a coexistence of two subgroups of non-transfused patients based on inclusion of patients with smaller clones.

Published data consistently demonstrate the occurrence of common PNH-related signs and symptoms and TE across all clone sizes. The substantial number of patients with a clone size <10% experiencing symptoms such as fatigue, abdominal pain, chest pain, and hemoglobinuria as well as TE, indicates that clone size alone is not a good indicator of burden of disease.

Table 4 summarizes the changes in FACIT-Fatigue and EORTC fatigue scores over time. The number of patients for which there were follow-up data remained the same as in the previously reported results for Eculizumab group. Data were available for an additional three patients in the No Eculizumab group. Median time from baseline to last available assessment increased from 0.9 to 1.5 years in the Eculizumab group, and from 1.4 to 1.7 years in the No Eculizumab group.

In the Eculizumab group, there was a substantial improvement in the fatigue score measured by both the FACIT-Fatigue instrument and the fatigue component of the EORTC QLQ-C30 questionnaire, from baseline to last available assessment. This improvement was clinically meaningful and statistically significant compared to fatigue levels reported by patients who did not receive eculizumab and were maintained on supportive therapy. In the Eculizumab group, 74% of the patients had a clinically meaningful improvement in FACIT-Fatigue score (at least 4 points) compared to only 27% in the No Eculizumab group. A similar clinically meaningful improvement in EORTC fatigue score (at least 10 points) was reported in 84% of the Eculizumab group compared to only 33% in the No Eculizumab group.

TABLE 4
PNH Registry Analysis—Change in Fatigue Scores from Baseline to
Last Available Assessment

| | | | No | |
|---|---|---|---|---|
| | Eculizumab | | Eculizumab | |
| | No | | No | |
| | Transfusion | | Transfusion | |
| | (N = 45) | | (N = 144) | |
| N | 19 | | 69 | |
| Change in N from 1 Jul. 2013 | No change | | +3 | |
| data extract | | | | |
| Median (min, max) assessment | 1.5 | (0.1, 3.9) | 1.7 | (0.4, 6.3) |
| time (years) | | | | |
| FACIT-Fatigue | | | | |
| Median (min, max) change in | 7.0 | (−5.0, 36.0) | 0.0 | (−34.0, 35.0) |
| FACIT-fatigue score^{a)} at last | | | | |
| available assessment | | | | |
| FACIT-Fatigue score^{a)} at last | 44.0 | (19.0, 52.0) | 40.5 | (6.0, 52.0) |
| assessment, median (min, max) | | | | |
| Patients with clinically | 14/19 | (73.7) | 18/66 | (27.3) |
| meaningful improvement^{b)}, n | | | | |
| EORTC-Fatigue | | | | |
| Median (min, max) change in | −22.2 | (−66.7, 22.2) | 0.0 | (−100.0, 55.6) |
| EORTC fatigue score^{c)} at last | | | | |
| available assessment | | | | |
| EORTC fatigue score^{c)} at last | 22.2 | (0.0, 100.0) | 33.3 | (0.0, 100.0) |
| assessment, median (min, max) | | | | |
| Patients with clinically | 16/19 | (84.2) | 23/69 | (33.3) |
| meaningful improvement^{d)}, n | | | | |

^{a)}higher scores indicate less fatigue
^{b)}Increase by at least 4 points (Cella, D. et al., J. Pain Symptom Manage., 24:547-61, 2002)
^{c)}higher scores indicate more fatigue
^{d)}decrease by at least 10 points (Cocks, K. et al., Eur. J. Cancer, 44:1793-8, 2008)

Table 5 summarizes the changes in hemoglobin levels over time. Patients without a history of transfusion had varying amounts of anemia at baseline. The median hemoglobin level, overall, was somewhat lower among patients who received eculizumab compared to those who were treated with supportive care only (No Eculizumab). At six months, there was a substantial increase in median hemoglobin in the Eculizumab group, but despite this improvement, residual anemia persisted for some patients, with a median hemoglobin of 111 g/L for the group. In both groups, the hemoglobin level remained stable after month six. These data, in conjunction with the LDH and fatigue data presented above, provide evidence that fatigue in patients with PNH is independent of improvement in hemoglobin levels, and improvement in hemoglobin levels is not an indicator of the underlying intravascular hemolysis, as evident in the No Eculizumab patients whose LDH levels remained elevated over time.

TABLE 5
PNH Registry Analysis - Hemoglobin (g/L) During Follow-Up

| | Eculizumab | | | No Eculizumab | | |
|---|---|---|---|---|---|---|
| | No Transfusion | | | No Transfusion | | |
| | (N = 45) | | | (N = 144) | | |
| | Median (min, | | | Median (min, | | |
| | max) | N | Change in N | max) | N | Change in N |
| Baseline | 100.0 (59.0, | 45 | No change | 116.5 (12.8, | 144 | No change |
| | 160.0) | | | 214.3) | | |
| 6 month | 111.6 (73.0, | 40 | +1 | 121.5 (73.0, | 108 | +2 |
| | 148.0) | | | 161.1) | | |
| 12 month | 111.0 (84.0, | 33 | +6 | 124.0 (74.0, | 109 | +12 |
| | 142.0) | | | 157.9) | | |
| 18 month | 113.0 (73.0, | 31 | +13 | 123.0 (75.7, | 85 | +19 |
| | 143.0) | | | 170.8) | | |
| 24 month | 111.0 (85.4, | 21 | +9 | 122.0 (70.0, | 61 | +18 |
| | 140.0) | | | 158.0) | | |

Table 6 and Table 7 summarize serum creatinine and eGFR during follow up. During follow-up, the renal function measured by serum creatinine and eGFR remained stable. CKD assessment was available for a limited number of patients making interpretation of the data difficult, however it is noteworthy that there were no patients whose CKD stage worsened at last available assessment relative to baseline in the Eculizumab group, whereas 9/28 (32%) patients in the No Eculizumab group worsened.

TABLE 6
PNH Registry Analysis - Serum Creatinine (□mol/L) During Follow-Up

| | Eculizumab | | | No Eculizumab | | |
|---|---|---|---|---|---|---|
| | No Transfusion | | | No Transfusion | | |
| | N = 45 | | | N = 144 | | |
| | Median (min, | | | Median (min, | | |
| | max) | N | Change in N | max) | N | Change in N |
| Baseline | 62.4 (35.0, 147.0) | 42 | No change | 70.7 (0.1, 180.0) | 131 | No change |
| 6 month | 70.7 (40.0, 139.0) | 38 | No change | 70.7 (38.0, | 101 | +1 |
| | | | | 459.7) | | |
| 12 month | 67.5 (44.2, 112.0) | 30 | +5 | 71.0 (42.0, | 94 | +11 |
| | | | | 132.6) | | |
| 18 month | 70.7 (44.2, 123.8) | 27 | +11 | 71.0 (41.0, 132.6) | 73 | +15 |
| 24 month | 66.8 (41.0, 123.8) | 18 | +7 | 71.0 (37.0, | 55 | +18 |
| | | | | 149.0) | | |

TABLE 7
PNH Registry Analysis - eGFR (mL/min/1.73 m²) During Follow-Up

| | Eculizumab | | | No Eculizumab | | |
|---|---|---|---|---|---|---|
| | No Transfusion | | | No Transfusion | | |
| | N = 45 | | | N = 144 | | |
| | Median | | | Median | | Change |
| | (min, max) | N | Change in N | (min, max) | N | in N |
| Base- | 109.6 (48.9, | 39 | No change | 99.0 | 126 | No |
| line | 137.8) | | | (31.6, | | change |
| | | | | 1010.2) | | |
| 6 | 105.3 (51.1, | 37 | No change | 100.7 | 99 | +1 |
| month | 128.4) | | | (11.3, | | |
| | | | | 144.1) | | |
| 12 | 104.6 (54.8, | 28 | +5 | 101.1 | 94 | +11 |
| month | 132.2) | | | (45.0, | | |
| | | | | 143.5) | | |
| 18 | 103.0 (54.3, | 25 | +11 | 101.9 | 73 | +15 |
| month | 131.7) | | | (44.9, | | |
| | | | | 135.3) | | |
| 24 | 110.5 (64.4, | 16 | +7 | 101.4 | 54 | +18 |
| month | 130.0) | | | (34.2, | | |
| | | | | 135.2) | | |

Table 8 summarizes patient reported outcomes as assessed by the EORTC QLQ-C30 scale. As noted above (Table 4), data were available for three additional patients in the No Eculizumab group; there was no change in patient numbers for the Eculizumab group. Treatment with eculizumab resulted in important improvement in quality of life measures as documented for Global Health, all five functioning scales and all but three of the other components (financial difficulty, diarrhea and constipation). Overall, more eculizumab treated patients had clinically meaningful improvement (improvement of ten or more points) compared to those who did not receive eculizumab.

TABLE 8
PNH Registry Analysis—Patient Reported Outcomes at Last Available
Assessment Using EORTC-QLQ-C30

| | Eculizumab | | No Eculizumab | |
|---|---|---|---|---|
| | No Transfusion | | No Transfusion | |
| | (N = 45) | | (N = 144) | |
| | Patients with | | Patients with | |
| | clinically | | clinically | |
| | meaningful | Mean (SD) | meaningful | Mean (SD) |
| | improvement^{a)}, | change from | improvement^{a)}, | change from |
| | n/N | baseline | n/N | baseline |
| Global | 8/17 (47.1) | 15.2 (23.61) | 15/67 (22.4) | −0.5 (21.61) |
| Health | | | | |
| Emotional | 13/19 (68.4) | 24.9 (25.87) | 22/69 (31.9) | 5.3 (28.19) |
| functioning | | | | |
| Social | 10/10 (52.6) | 21.9 (31.94) | 20/69 (29.0) | 4.3 (22.08) |
| functioning | | | | |
| Cognitive | 11/19 (57.9) | 18.4 (28.81) | 19/69 (27.5) | 2.4 (20.47) |
| functioning | | | | |
| Role | 9/19 (47.4) | 24.6 (33.04) | 13/69 (18.8) | −0.2 (27.04) |
| functioning | | | | |
| Physical | 8/19 (42.1) | 10.5 (17.40) | 6/69 (8.7) | 0.2 (8.88) |
| functioning | | | | |
| Dyspnea | 8/19 (42.1) | −21.1 (33.72) | 11/69 (15.9) | −1.0 (26.18) |
| Nausea/ | 9/19 (47.4) | −13.2 (25.20) | 10/69 (14.5) | −1.4 (12.04) |
| vomiting | | | | |
| Insomnia | 7/19 (36.8) | −12.3 (22.80) | 13/69 (18.8) | 5.3 (32.65) |
| Pain | 7/19 (36.8) | −10.5 (23.05) | 13/69 (18.8) | −2.4 (25.13) |
| Appetite | 6/19 (31.6) | −15.8 (32.14) | 10/69 (14.5) | −3.4 (20.73) |
| loss | | | | |
| Financial | 3/19 (15.8) | −5.3 (27.81) | 11/69 (15.9) | −5.3 (29.50) |
| difficulty | | | | |
| Diarrhea | 2/18 (11.1) | −1.9 (13.87) | 12/68 (17.6) | −2.0 (25.68) |
| Constipation | 2/19 (10.5) | −3.5 (10.51) | 10/69 (14.5) | 0.0 (24.92) |

^{a)}Increase by at least 10 points.

Patients treated with eculizumab demonstrated statistically and clinically significant improvement in LDH levels, returning to and maintaining near normal levels of LDH, indicating sustained reduction and control of complement-mediated intravascular hemolysis. These results are in striking contrast to the patients who did not receive eculizumab whose LDH levels remain elevated at levels above 1.5×ULN, and therefore indicate ongoing increased risk for the associated disabling morbidities and premature mortality associated with PNH.

The updated data confirms that eculizumab treatment results in a statistically and clinically significant improvement in fatigue scores, as measured by both the FACIT-Fatigue and EORTC scales, in patients with PNH and no history if transfusion. These data are consistent with what was reported for patients in our registrational trials (TRIUMPH and SHEPHERD). Clinically meaningful improvements continued to be reported in numerous quality of life measures in patients treated with eculizumab. Notably, improvement was not observed in the No Eculizumab patients. These additional longitudinal data and the uniformly consistent results over time provide further evidence of the robustness of the data from the PNH Registry. Overall, the results demonstrate that control of intravascular hemolysis is essential for the improvement of symptoms in patients with PNH, and establish the benefit of eculizumab in the treatment of patients with LDH≥1.5×ULN, regardless of transfusion history. Transfusion requirements do not provide a valuable measure of the severity of disease in patients with PNH. Instead, severity can be assessed by the level of chronic hemolytic anemia (e.g., intravascular hemolysis), which is measured by LDH.

There were 189 patients with no history of transfusion and an LDH≥1.5×ULN that were included in the PNH Registry. The majority (92%) of these patients had symptomatic chronic hemolytic anemia at baseline defined as the physician having reported the presence of abdominal pain, shortness of breath, dysphagia, erectile dysfunction, fatigue or anemia (defined as hemoglobin level <100 g/L, CTCAE version 4.0). Specifically, 43/45 (96%) patients in the Eculizumab group and 130/144 (90%) patients in the No Eculizumab group had at least one of the above listed symptoms at baseline. These numbers further support the relevance of LDH for identifying patients with clinically meaningful burden of disease.

Table 9 and Table 10 summarize the primary and secondary outcomes from an analysis of this subset of patients. These data demonstrate that transfusion history is not an indicator of burden of disease in patients with PNH. Furthermore, eculizumab provides a substantial benefit, resulting in a statistically significant reduction in intravascular hemolysis (p<0.001 for change in LDH from baseline at 6 months) and fatigue (p=0.014 and p=0.028 for change in scores from baseline to last available assessment for FACIT-Fatigue and EORTC fatigue, respectively).

TABLE 9
PNH Registry—LDH in Patients With Symptomatic
Chronic Hemolytic Anemia*

| | Eculizumab | | No Eculizumab | |
|---|---|---|---|---|
| | No Transfusion | | No Transfusion | |
| | (N = 43) | | (N = 130) | |
| | | Median | | Median |
| | N | (min, max) | N | (min, max) |
| LDH value | | | | |
| (U/L) | | | | |
| Baseline | 43 | 1447.0 | 130 | 1116.0 |
| | | (301.0, 4661.0) | | (360.0, 4893.0) |
| 6 month | 36 | 305.5 | 91 | 1024 |
| | | (142.0, 1497.0) | | (237.0, 5212.0) |
| LDH ratio | | | | |
| (×ULN) | | | | |
| Baseline | 43 | 4.6 (1.5, 15.8) | 130 | 3.6 (1.5, 11.4) |
| 6 month | 34 | 1.1 (0.7, 3.4) | 87 | 3.4 (0.9, 9.5) |

*Symptomatic chronic hemolytic anemia defined as the physician having reported the presence of abdominal pain, shortness of breath, dysphagia, erectile dysfunction, fatigue or anemia

TABLE 10
PNH Registry—Fatigue Scores in Patients With Symptomatic Chronic
Hemolytic Anemia*

| | Eculizumab | | No Eculizumab | |
|---|---|---|---|---|
| | No Transfusion | | No Transfusion | |
| | (N = 43) | | (N = 130) | |
| | | Median | | Median |
| | N | (min, max) | N | (min, max) |
| Median (min, max) | 19 | 7.0 (−5.0, 36.0) | 61 | 0.0 (−34.0, 35.0) |
| change in FACIT- | | | | |
| Fatigue score^{a)} | | | | |
| FACIT-Fatigue score | 31 | 44.0 (19.0, 52.0) | 97 | 39.0 (6.0, 52.0) |
| at last assessment | | | | |
| Median (min, max) | 19 | −22.2 (−66.7, 22.2) | 61 | 0.0 (−100.0, 55.6) |
| change EORTC | | | | |
| fatigue score^{b)} at last | | | | |
| assessment | | | | |
| EORTC fatigue score | 31 | 22.2 (0.0, 100.0) | 97 | 33.3 (0.0, 100.0) |
| at last assessment | | | | |

^{a)}FACIT-Fatigue scale range is 0-52 with higher scores better (less fatigue)
^{b)}EORTC fatigue scale range is 0-100 with higher scores worse (more fatigue)
*Symptomatic chronic hemolytic anemia defined as the physician having reported the presence of abdominal pain, shortness of breath, dysphagia, erectile dysfunction, fatigue or anemia

Similar to the results for the patients in the PNH Registry with no history of transfusion, among the 22 SHEPHERD patients with 0-1 transfusions in the 12 months prior to enrollment, 21/22 (95%) had either fatigue, shortness of breath and/or anemia (as defined by hemoglobin <100 g/L). In the ≥2 Transfusions subgroup, 64/75 (85%) patients presented with at least one of these symptoms at baseline (Table 11).

Table 11 and Table 12 provide a summary of the primary and secondary outcomes for the subgroups of patients from the SHEPHERD sub-analysis. Both subgroups, regardless of transfusion history, demonstrated intravascular hemolysis measured by LDH levels and fatigue at baseline, which was significantly reduced after treatment with eculizumab.

TABLE 11
SHEPHERD—LDH in Patients With Symptomatic
Chronic Hemolytic Anemia*

| | SHEPHERD | | SHEPHERD | |
|---|---|---|---|---|
| | Eculizumab | | Eculizumab | |
| | 0-1 Transfusion | | ≥2 Transfusions | |
| | (N = 21) | | (N = 64) | |
| LDH value | | Median | | Median |
| (U/L) | N | (min, max) | N | (min, max) |
| Baseline | 21 | 2030.0 | 64 | 2162.5 |
| | | (824.0, 3851.0) | | (694.0, 5245.0) |
| 6 month | 21 | 260.0 | 63 | 264.0 |
| | | (164.0, 1079.0) | | (98.0, 1494.0) |

*Symptomatic chronic hemolytic anemia defined as fatigue (defined as “quite a bit” or “very much”; score of 3 and 4, respectively), shortness of breath or anemia

TABLE 12
SHEPHERD—Fatigue Scores in Patients With Symptomatic
Chronic Hemolytic Anemia*

| | SHEPHERD | | | SHEPHERD | | |
|---|---|---|---|---|---|---|
| | Eculizumab | | | Eculizumab | | |
| | 0-1 Transfusion | | | ≥2 Transfusions | | |
| | (N = 21) | | | (N = 64) | | |
| | | Median | | | Median | |
| | N | (min, max) | | N | (min, max) | |
| Median (min, | 21 | 16.0 | (0.0, 37.0) | 63 | 8.0 | (−8.0, 44.0) |
| max) change | | | | | | |
| from baseline in | | | | | | |
| FACIT-Fatigue | | | | | | |
| score^{a)} at last | | | | | | |
| assessment | | | | | | |
| FACIT-Fatigue | 21 | 41.0 | (21.0, 52.0) | 63 | 45.0 | (19.0, 52.0) |
| score at last | | | | | | |
| assessment, | | | | | | |
| median (min, | | | | | | |
| max) | | | | | | |
| Median (min, | 21 | −33.3 | (−88.9, 0.0) | 63 | −22.2 | (−100.0, 22.2) |
| max) change | | | | | | |
| from baseline | | | | | | |
| in EORTC | | | | | | |
| fatigue score^{b)} at | | | | | | |
| last assessment | | | | | | |
| EORTC fatigue | 21 | 22.2 | (0.0, 66.7) | 63 | 22.2 | (0.0, 77.8) |

score at last
assessment,
median (min,
max)

^{a)}FACIT-Fatigue scale range is 0-52 with higher scores better (less fatigue)
^{b)}EORTC fatigue scale range is 0-100 with higher scores worse (more fatigue)
*Symptomatic chronic hemolytic anemia defined as fatigue (defined as “quite a bit” or “very much”; score of 3 and 4, respectively), shortness of breath or anemia

Overall, almost all the patients in the PNH Registry analysis with no history of transfusion exhibited symptomatic chronic hemolytic anemia as defined above. Similarly, all but one of the patients in the SHEPHERD subgroup with 0-1 transfusions in the 12 months prior to enrollment had experienced either fatigue, shortness of breath and/or anemia. Together these data support that LDH≥1.5×ULN is the most relevant marker to identify burden of disease characterized as symptomatic chronic hemolytic anemia in patients with PNH, independent of hemoglobin levels and associated transfusions.

A responder analysis for the FACIT-fatigue data was performed. It is important to keep in mind that limitations exist when collapsing a continuous variable such as fatigue score into categories of severity since this would imply available information relating the measurement tool to the severity categories. A published approach provides clinical severity thresholds for normal, mild, moderate and severe fatigue on the PROMIS fatigue metric (Lai, J. et al., Psychooncology, 23:1133-41, 2014). Another approach links the FACIT-Fatigue to the PROMIS Fatigue metric. Based on these two approaches (Table 5 from Lai et al.), and FIG. 3 from (Cella, D. et al., Qual. Life Res., 23:2651-61, 2014), the following score ranges are associated with each severity level:
Normal: 42-52
Mild: 35-41
Moderate: 6-34
Severe: 0-5

Table 13 displays FACIT-Fatigue results for the Eculizumab group with PNH at baseline and at last available assessment (n=19). Among these patients, 68.4% (n=13) reported mild or moderate fatigue at baseline. At last available assessment, notably fewer patients reported mild or moderate fatigue (31.6%, n=6). Of the 13 patients in the group who reported mild or moderate fatigue at baseline, improvement was observed in 61.5% (n=8).

TABLE 13
Categorical Analysis of Change in FACIT-Fatigue Among Patients in the Eculizumab
No Transfusion Group (N = 19)

| | | Last Available Assessment | | | | |
|---|---|---|---|---|---|---|
| | | Normal | Mild | Moderate | Severe | Baseline |
| | | (42-52) | (35-<42) | (6-<35) | (0-<6) | Total |
| Baseline | Normal (42-52) | 5 (83.3%) | 1 (16.7%) | 0 (0.0%) | 0 (0.0%) | 6 (31.6%) |
| | Mild (35-<42) | 2 (66.7%) | 1 (33.3%) | 0 (0.0%) | 0 (0.0%) | 3 (15.8%) |
| | Moderate (6-<35) | 6 (60.0%) | 1 (10.0%) | 3 (30.0%) | 0 (0.0%) | 10 (52.6%) |
| | Severe (0-<6) | 0 (NA) | 0 (NA) | 0 (NA) | 0 (NA) | 0 (0.0%) |

| | | | | | |
|---|---|---|---|---|---|
| Last Available Total | 13 (68.4%) | 3 (15.8%) | 3 (15.8%) | 0 (0.0%) | 19 (100%) |

Table 14 shows FACIT-Fatigue results for patients in the No Eculizumab group at baseline and at last available assessment (n=69). There were 52.2% (n=36) who reported mild or moderate fatigue at baseline. At last available assessment, approximately the same proportion of patients reported mild or moderate fatigue as at baseline (49.2%, n=34). Of the 36 patients in the No Eculizumab group who reported mild or moderate fatigue at baseline, only a low proportion (25%, n=9) improved to normal fatigue at last available assessment.

TABLE 14
Categorical Analysis of Change in FACIT-Fatigue Among Patients in the No
Eculizumab No Transfusion Group (N = 69)

| | | Last Available Assessment | | | | |
|---|---|---|---|---|---|---|
| | | Normal | Mild | Moderate | Severe | Baseline |
| | | (42-52) | (35-<42) | (6-<35) | (0-<6) | Total |
| Baseline | Normal (42-52) | 26 (78.8%) | 3 (9.1%) | 4 (12.1%) | 0 (0.0%) | 33 (47.8%) |
| | Mild (35-<42) | 4 (28.6%) | 3 (21.4%) | 7 (50.0%) | 0 (0.0%) | 14 (20.3%) |
| | Moderate (6-<35) | 5 (22.7%) | 3 (13.6%) | 14 (63.6%) | 0 (0.0%) | 22 (31.9%) |
| | Severe (0-<6) | 0 (NA) | 0 (NA) | 0 (NA) | 0 (NA) | 0 (0.0%) |

| | | | | | |
|---|---|---|---|---|---|
| Last Available | 35 (50.7%) | 9 (13.0%) | 25 (36.2%) | 0 (0.0%) | 69 (100%) |
| Total | | | | | |

The updated PNH Registry analysis for FACIT-Fatigue scores demonstrates clinically meaningful and statistically significant improvement in fatigue among the Eculizumab No Transfusion patients. This additional responder analysis further demonstrates that treating patients with no history of transfusion with eculizumab results in a clinically meaningful improvement in fatigue from baseline to last available assessment.

TABLE 4
Efficacy Outcomes (LDH level and FACIT-Fatigue)
in PNH patients without a history of transfusion

| | M07-001 | |
|---|---|---|
| | No Eculizumab | Eculizumab |
| | No | No |
| Parameter | transfusion | transfusion |
| LDH level at baseline | N = 144 | N = 45 |
| (median, U/L) | 1095 | 1431 |
| LDH level at 6 months | N = 102 | N = 38 |
| (median, U/L) | 1033 | 294 |
| FACIT-Fatigue score at | N = 88 | N = 25 |
| baseline (median) | 39 | 32 |
| FACIT-Fatigue score at | N = 108 | N = 33 |
| last follow-up (median) | 41 | 44 |

### OTHER EMBODIMENTS

It is to be understood that while the invention has been described in conjunction with the detailed description thereof, the foregoing description is intended to illustrate and not limit the scope of the invention, which is defined by the scope of the appended claims. The materials, methods, and examples are illustrative only and not intended to be limiting. All publications, patent applications, patents, sequences, database entries and other references cited and described herein are incorporated by reference in their entireties. Other aspects, advantages, and modifications are within the scope of the following claims.

SEQUENCE SUMMARY

SEQ ID NO: 1
amino acid sequence of heavy chain CDR1 of eculizumab (as defined
under combined Kabat-Chothia definition)
GYIFSNYWIQ
SEQ ID NO: 2
amino acid sequence of heavy chain CDR2 of eculizumab (as defined
under Kabat definition)
EILPGSGSTEYTENFKD
SEQ ID NO: 3
amino acid sequence of the heavy chain CDR3 of eculizumab (as
defined under combined Kabat definition).
YFFGSSPNWYFDV
SEQ ID NO: 4
amino acid sequence of the light chain CDR1 of eculizumab (as
defined under Kabat definition)
GASENIYGALN
SEQ ID NO: 5
amino acid sequence of light chain CDR2 of eculizumab
(as defined under Kabat definition)
GATNLAD
SEQ ID NO: 6
amino acid sequence of light chain CDR3 of eculizumab (as defined
under Kabat definition)
QNVLNTPLT
SEQ ID NO: 7
amino acid sequence of heavy chain variable region of eculizumab
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCARY
FFGSSPNWYFDVWGQGTLVTVSS
SEQ ID NO: 8
amino acid sequence of light chain variable region of eculizumab,
BNJ441 antibody, and BNJ421 antibody
DIQMTQSPSSLSASVGDRVTITCGASENIYGALNWYQQKPGKAPKLLIYGA
TNLADGVPSRFSGSGSGTDFTLTISSLQPEDFATYYCQNVLNTPLTFGQGTK
VEIK
SEQ ID NO: 9
amino acid sequence of heavy chain constant region of eculizumab
and BNJ421 antibody
ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKC
CVECPPCPAPPVAGPSVFLFPPKPKDTLMISRTPEVTCVVVDVSQEDPEVQF
NWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK
SEQ ID NO: 10
amino acid sequence of entire heavy chain of eculizumab
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 11
amino acid sequence of entire light chain of eculizumab, BNJ441
antibody, and BNJ421 antibody
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPV
TKSFNRGEC
SEQ ID NO: 12
amino acid sequence of heavy chain variable region of BNJ441
antibody and BNJ421 antibody
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS
SEQ ID NO: 13
amino acid sequence of heavy chain constant region of BNJ441
antibody
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDPE
VQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
amino acid sequence of entire heavy chain of BNJ441 antibody
QVQLVQSGAEVKKPGASVKVSCKASGHIFSNYWIQWVRQAPGQGLEWM
GEILPGSGHTEYTENFKDRVTMTRDTSTSTVYMELSSLRSEDTAVYYCAR
YFFGSSPNWYFDVWGQGTLVTVSS ASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSV [symbol] HEALH [symbol] HYTQKSLSLSLGK
SEQ ID NO: 15
amino acid sequence of IgG2 heavy chain constant region variant
comprising YTE substitutions
ASTKGPSVFPLAPCSRSTSESTAALGCLVKDYFPEPVTVSWNSGALTSGVH
TFPAVLQSSGLYSLSSVVTVTSSNFGTQTYTCNVDHKPSNTKVDKTVERKC
CVECPPCPAPPVAGPSVFLFPPKPKDTLYITREPEVTCVVVDVSHEDPEVQF
NWYVDGMEVHNAKTKPREEQFNSTFRVVSVLTVVHQDWLNGKEYKCKV
SNKGLPAPIEKTISKTKGQPREPQVYTLPPSREEMTKNQVSLTCLVKGFYP
SDIAVEWESNGQPENNYKTTPPMLDSDGSFFLYSKLTVDKSRWQQGNVF
SCSVMHEALHNHYTQKSLSLSPGK
SEQ ID NO: 16
amino acid sequence of entire heavy chain of eculizumab variant
comprising heavy chain constant region depicted in SEQ ID NO: 15
(above)
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVTSSNF
GTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK
SEQ ID NO: 17
amino acid sequence of light chain CDR1 of eculizumab (as defined
under Kabat definition) with glycine to histidine substitution at
position 8 relative to SEQ ID NO: 4
GASENIYHALN
SEQ ID NO: 18
depicts amino acid sequence of heavy chain CDR2 of eculizumab in
which serine at position 8 relative to SEQ ID NO: 2 is substituted
with histidine
EILPGSGHTEYTENFKD
SEQ ID NO: 19
amino acid sequence of heavy chain CDR1 of eculizumab in which
tyrosine at position 2 (relative to SEQ ID NO: 1) is substituted
with histidine
GHIFSNYWIQ
SEQ ID NO: 20
amino acid sequence of entire heavy chain of BNJ421 antibody
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTLMIS
RTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSV [symbol] HEALH [symbol] HYTQKSLSLSLGK

1. A method of reducing intravascular hemolysis in a human patient having Paroxysmal Nocturnal Hemoglobinuria (PNH), the method comprising administering an effective amount an anti-C5 antibody, or antigen binding fragment thereof, comprising:
(a) CDR1, CDR2, and CDR3 heavy chain sequences as set forth in SEQ ID NOs:1, 2, and 3, respectively, and CDR1, CDR2, and CDR3 light chain sequences as set forth in SEQ ID NOs:4, 5, and 6, respectively, or
(b) CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively,
to the patient, wherein prior to treatment the patient patient is determined to have high disease activity as determined by a lactate dehydrogenase concentration of about ≥1.5× upper limit of normal (ULN), and wherein the patient does not exhibit symptoms of PNH prior to treatment.

2. (canceled)

3. The method of claim 1, wherein the anti-C5 antibody, or antigen binding fragment thereof, comprises CDR1, CDR2, and CDR3 heavy chain sequences as set forth in SEQ ID NOs:19, 18, and 3, respectively, CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

4. A method of reducing intravascular hemolysis in a human patient having Paroxysmal Nocturnal Hemoglobinuria (PNH), the method comprising:
(i) selecting a patient who has a lactate dehydrogenase concentration of about ≥1.5× upper limit of normal (ULN) and does not exhibit symptoms of PNH prior to treatment, from a subpopulation of PNH patients; and
(ii) administering to the patient an anti-C5 antibody, or antigen binding fragment thereof, comprising:
(a) CDR1, CDR2, and CDR3 heavy chain sequences as set forth in SEQ ID NOs:1, 2, and 3, respectively, and CDR1, CDR2, and CDR3 light chain sequences as set forth in SEQ ID NOs:4, 5, and 6, respectively, or
(b) CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively.

5. (canceled)

6. The method of claim 4, wherein the anti-C5 antibody, or antigen binding fragment thereof, comprises CDR1, CDR2, and CDR3 heavy chain sequences as set forth in SEQ ID NOs:19, 18, and 3, respectively, CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

7. The method of claim 3, wherein the anti-C5 antibody, or antigen-binding fragment thereof, comprises a heavy chain variable region depicted in SEQ ID NO:12 and a light chain variable region depicted in SEQ ID NO:8.

8. The method of claim 3, wherein the anti-C5 antibody, or antigen-binding fragment thereof, comprises, wherein the anti-C5 antibody, or antigen-binding fragment thereof, further comprises a heavy chain constant region depicted in SEQ ID NO:13.

9. The method of claim 3 or 6, wherein the antibody, or antigen-binding fragment thereof, comprises a heavy chain polypeptide comprising the amino acid sequence depicted in SEQ ID NO:14 and a light chain polypeptide comprising the amino acid sequence depicted in SEQ ID NO:11.

10. The method of claim 1, wherein the patient is determined to have a lactate dehydrogenase concentration of about 1.5× to about 3.5×ULN.

11. The method of claim 1, wherein the patient has never had a blood transfusion.

12. The method of claim 1, wherein the patient does not have a history of thrombosis and/or fatigue.

13. The method of claim 1, wherein the treated patient experiences a return to normal lactate dehydrogenase concentration within six months of treatment with the anti-C5 antibody, or antigen binding fragment thereof.

14. The method of claim 1, wherein treatment begins with an initial phase comprising administering 600 mg of the anti-C5 antibody, or antigen binding fragment thereof, once a week for 4 weeks.

15. The method of claim 14, wherein the initial phase of treatment is followed by a maintenance phase comprising administering 900 mg of the anti-C5 antibody, or antigen binding fragment thereof, during the fifth week.

16. The method of claim 15, wherein the maintenance phase is followed by administration of 900 mg of the anti-C5 antibody, or antigen binding fragment thereof, every 14±2 days.

17. The method of claim 1, wherein the patient is a pediatric patient having a body weight of between about 30 and about 40 kg, and treatment begins with an initial phase comprising administering 600 mg of the anti-C5 antibody, or antigen binding fragment thereof, once a week for 2 weeks.

18. The method of claim 17, wherein the initial phase of treatment is followed by a maintenance phase comprising administering 900 mg of the anti-C5 antibody, or antigen binding fragment thereof, during the third week.

19. The method of claim 18 wherein the maintenance phase is followed by administration of 900 mg of the anti-C5 antibody, or antigen binding fragment thereof, every 2 weeks.

20. The method of claim 1, wherein the patient is a pediatric patient having a body weight of between about 20 and about 30 kg, and treatment begins with an initial phase comprising administering 600 mg of the anti-C5 antibody, or antigen binding fragment thereof, once a week for 2 weeks.

21. The method of claim 20, wherein the initial phase of treatment is followed by a maintenance phase comprising administering 600 mg of the anti-C5 antibody, or antigen binding fragment thereof, during the third week.

22. The method of claim 21, wherein the maintenance phase is followed by administration of 600 mg of the anti-C5 antibody, or antigen binding fragment thereof, every 2 weeks.

23. The method of claim 1, wherein the patient is a pediatric patient having a body weight of between about 10 and about 20 kg, and treatment begins with an initial phase comprising administering 600 mg of the anti-C5 antibody, or antigen binding fragment thereof, once a week for 1 week.

24. The method of claim 23, wherein the initial phase of treatment is followed by a maintenance phase comprising administering 300 mg of the anti-C5 antibody, or antigen binding fragment thereof, during the second week.

25. The method of claim 24, wherein the maintenance phase is followed by administration of 300 mg of the anti-C5 antibody, or antigen binding fragment thereof, every 2 weeks.

26. The method of claim 1, wherein the patient is a pediatric patient having a body weight of between about 5 and about 10 kg, and treatment begins with an initial phase comprising administering 300 mg of the anti-C5 antibody, or antigen binding fragment thereof, once a week for 1 week.

27. The method of claim 26, wherein the initial phase of treatment is followed by a maintenance phase comprising administering 300 mg of the anti-C5 antibody, or antigen binding fragment thereof, during the second week.

28. The method of claim 27, wherein the maintenance phase is followed by administration of 300 mg of the anti-C5 antibody, or antigen binding fragment thereof, every 3 weeks.

29. The method of claim 1, wherein the anti-C5 antibody, or antigen binding fragment thereof, is administered through intravenous infusion.


SEQUENCE LISTING (20 sequences)

SEQ ID NO: 1 - protein, 10 aa, Artificial Sequence
  source: /note=“Description of Artificial Sequence       Synthetic peptide”
GYIFSNYWIQ

SEQ ID NO: 2 - protein, 17 aa, Artificial Sequence
  source: /note=“Description of Artificial Sequence       Synthetic peptide”
EILPGSGSTEYTENFKD

SEQ ID NO: 3 - protein, 13 aa, Artificial Sequence
  source: /note=“Description of Artificial Sequence       Synthetic peptide”
YFFGSSPNWYFDV

SEQ ID NO: 4 - protein, 11 aa, Artificial Sequence
  source: /note=“Description of Artificial Sequence       Synthetic peptide”
GASENIYGALN

SEQ ID NO: 5 - protein, 7 aa, Artificial Sequence
  source: /note=“Description of Artificial Sequence       Synthetic peptide”
GATNLAD

SEQ ID NO: 6 - protein, 9 aa, Artificial Sequence
  source: /note=“Description of Artificial Sequence       Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence       Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence       Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence       Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence       Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence       Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence       Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence       Synthetic peptide”
GASENIYHALN

SEQ ID NO: 18 - protein, 17 aa, Artificial Sequence
  source: /note=“Description of Artificial Sequence       Synthetic peptide”
EILPGSGHTEYTENFKD

SEQ ID NO: 19 - protein, 10 aa, Artificial Sequence
  source: /note=“Description of Artificial Sequence       Synthetic peptide”
GHIFSNYWIQ

SEQ ID NO: 20 - protein, 448 aa, Artificial Sequence
  source: /note=“Description of Artificial Sequence       Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK